(19) United States
(12) Patent Application Publication
(10) Pub. No.: US 2007/0149489 A1
(43) Pub. Date: Jun. 28, 2007

(54) Preparation of paricalcitol

(76) Inventors: Anchel Schwartz, Rehovot (IL); Alexei Ploutno, Bat-Yam (IL); Koby Wolfman, Petah Tikva (IL)

Correspondence Address: KENYON & KENYON LLP, ONE BROADWAY, NEW YORK, NY, 10004, US

(21) Appl. No.: 11/489,148

(22) Filed: Jul. 18, 2006

Related U.S. Application Data
(60) Provisional application No. 60/700,477, filed on Jul. 18, 2005.

(51) Int. Cl. A61K 31/59; C07C 401/00

(52) U.S. Cl. 514/167; 552/653

(57) ABSTRACT

The present invention is directed to a novel process for preparing Paricalcitol wherein Paricalcitol, dissolved in a solvent, is precipitated from a concentrated or seeded solution.

### CROSS REFERENCE TO RELATED APPLICATIONS

The present application claims the benefit of the following U.S. Provisional Patent Application No. 60/700,477 filed Jul. 18, 2005. The contents of which are incorporated herein by reference.

### FIELD OF INVENTION

The present invention is directed to a process for preparing Paricalcitol.

### BACKGROUND OF THE INVENTION

Vitamin D is a fat-soluble vitamin. It is found in food, but also can be formed in the body after exposure to ultraviolet rays. Vitamin D is known to exist in several chemical forms, each with a different activity. Some forms are relatively inactive in the body, and have limited ability to function as a vitamin. The liver and kidney help convert vitamin D to its active hormone form. The major biologic function of vitamin D is to maintain normal blood levels of calcium and phosphorus. Vitamin D aids in the absorption of calcium, helping to form and maintain healthy bones.

The 19-nor vitamin D analogue, Paricalcitol (I), is characterized by the following formula:

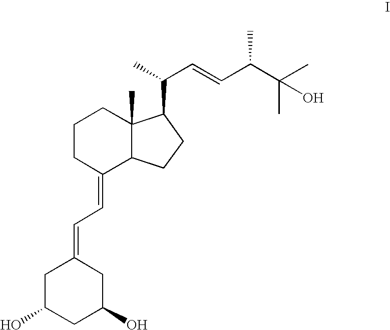

In the synthesis of vitamin D analogues, a few approaches to obtain a desired active compound have been outlined previously. One of the methods is the Wittig-Homer attachment of a 19-nor A-ring phosphine oxide to a key intermediate bicyclic-ketone of the Windaus-Grundmann type, to obtain the desired Paricalcitol, as is shown for example in U.S. Pat. Nos. 5,281,731 and 5,086,191 of DeLuca.

The synthesis of Paricalcitol requires many synthetic steps which produce undesired by-products. Therefore, the final product may be contaminated not only with a by-product derived from the last synthetic step of the process but also with compounds that were formed in previous steps. In the United States, the Food and Drug Administration guidelines recommend that the amounts of some impurities be limited to less than 0.1 percent.

U.S. Pat. Nos. 5,281,731 and 5,086,191 of DeLuca disclose a purification process of Paricalcitol by using a HPLC preparative method.

As the unwanted products have almost the same structure as the final product, it may difficult to get a sufficiently pure drug substance, vitamin D analogue, using this route to purify the drug substance. Moreover, the high polarity of Paricalcitol makes it very difficult to purify by HPLC and to recover the solid product. Furthermore, HPLC preparative methods are generally not applicable for use on industrial scale. There remains a need in the art to provide a method of preparing the vitamin D analogue Paricalcitol in a sufficiently pure form which is applicable for use on an industrial scale.

### SUMMARY OF THE INVENTION

In one aspect, the present invention provides a method for purifying Paricalcitol comprising the steps of

a) dissolving Paricalcitol in a solvent;

b) cooling the solution to form a precipitate; and

c) recovering the precipitate. Preferably the solvent is selected from the group consisting of a C₂-C₆ ether, a C₂-C₄ ester, a mixture of C₂-C₄ ester/H₂O, a C₃-C₅ ketone, a mixture of C₃-C₅ ketone/H₂O, a C₁-C₄ alcohol, a mixture of C₂-C₆ ether/C₃-C₅ ketone, a mixture of C₂-C₆ ether/C₂-C₄ ester, a mixture of C₂-C₆ ether/C₁-C₄ alcohol, acetonitrile, a mixture of acetonitrile/H₂O, and mixtures thereof, more preferably the solvent is selected from the group consisting of tert-butanol, acetone, acetone/H₂O, diethyl ether, ethyl acetate, ethyl acetate/H₂O, diethyl ether/acetone, acetonitrile, acetonitrile/H₂O, and mixtures thereof. Most preferably, the solvent is acetone.

### DETAILED DESCRIPTION OF THE INVENTION

The invention provides a process for purifying Paricalcitol. This process may be practiced without the need for an HPLC preparative method. The process of the invention may be easily applied to an industrial scale. Industrial scale process is that which prepares a batch of at least 5 g of the API, more preferably at least 10 g of the API.

During the preparation of Paricalcitol, various unwanted by-products may be formed, depending on the method employed for its preparation. One of the most common by-products is its C-24 isomer. Another common by-product is its C-14 epimer.

In one aspect, the present invention provides a method for purifying Paricalcitol comprising the steps of

a) dissolving Paricalcitol in a solvent;

b) cooling the solution to form a precipitate; and

c) recovering the precipitate.

The solvent for use in the method of the present invention is preferably selected from the group consisting of a C₂-C₆ ether, a C₂-C₄ ester, a mixture of C₂-C₄ ester/H₂O, a C₃-C₅ ketone, a mixture of C₃-C₅ ketone/H₂O, a C₁-C₄ alcohol, a mixture of C₂-C₆ ether/C₃-C₅ ketone, a mixture of C₂-C₆ ether/C₂-C₄ ester, a mixture of C₂-C₆ ether/C₁-C₄ alcohol, acetonitrile, a mixture of acetonitrile/H₂O, and mixtures thereof, more preferably the solvent is selected from the group consisting of tert-butanol, acetone, acetone/H₂O, diethyl ether, ethyl acetate, ethyl acetate/H₂O, diethyl ether/acetone, acetonitrile, acetonitrile/H₂O, and mixtures thereof. Most preferably, the solvent is acetone.

Preferably, the ratio between Paricalcitol and the solvent is about 1:150-1:450 g of Paricalcitol/ml of solvent, more preferably about 1:150-1:250 g of Paricalcitol/ml of solvent, most preferably about 1:150-1:200 g Paricalcitol/ml of solvent. In addition, the step of dissolving Paricalcitol in a solvent is preferably carried out at a temperature of about 25° C. to about 40° C., more preferably at a temperature of about 28° C. to about 34° C.

The solution is preferably filtered after the step of dissolving Paricalcitol in a solvent in the method of the present invention, to obtain a clear solution. The filtration removes solids that have not dissolved in the solvent.

Preferably, the solution is cooled to a temperature of about −45° C. to about −10° C., more preferably about −20° C. to about −15° C., most preferably to a temperature of about −18° C. However, some solvents suitable for use in the method of the present invention freeze at such low temperatures, for example (clean) tert-butanolf freezes at temperatures between 24° C. and 26° C. In such cases, the solution is cooled to a temperature above the freezing point so as to maintain the solution in liquid form. Therefore, when tert-butanol is used as a solvent in the method of the present invention, the solution is cooled to a temperature of about 25° C.-27° C.

In one embodiment of the present invention the solution is cooled at a controlled slow rate. Preferably, the solution is cooled at a rate of not more than about 8° C. per hour, more preferably not more than about 4° C. per hour. The cooling of the solution at a slow rate results in decreased amounts, less than about 5000 ppm, of residual solvent in the purified composition. Preferably, cooling the solution at a slow rate reduces the amount of residual solvent to about 800-1500 ppm.

The solution is cooled for a sufficient amount of time to obtain a desirable amount of solids. Preferably, the solution is cooled for a period of about 15 to about 24 hours, more preferably for a period of about 16 to about 20 hours. When tert-butanol is used as the solvent in the method of the present invention, the solution is cooled at a temperature of about 25° C.-27° C. for a period of about 1 to about 4 hours.

In the present invention dissolution of Paricalcitol in a solvent is preferably carried out in a sonicator. The use of sonication while dissolving Paricalcitol enables the use of relatively low amounts of solvent.

In another aspect of the present invention the method further comprises concentrating the solution of Paricalcitol in solvent from step a) before cooling the solution. Preferably, the solution is concentrated to obtain a ratio of about 1:100-1:120 g Paricalcitol/ml of solvent. Therefore, the solution is concentrated in the method of the present invention to reduce its volume to about 0.5 to about 0.9, preferably about 0.6 to about 0.8, times its original volume. Concentrating the solution in the method of the present invention may be carried out using methods know to those skilled in the art. Such methods of concentrating the solution include for example concentration by evaporation, filtration, and dialysis. When the solvent for dissolving Paricalcitol is a mixture of solvents as described above, concentrating the solution of dissolved Paricalcitol in the solvent mixture is optional.

In another aspect of the method of the present invention the method further comprises seeding the solution with crystals either before or during the step of cooling the solution. The solution may be seeded to promote crystallization. Crystals of Paricalcitol may be used as seeds. In one embodiment, both a seeding and a concentrating step is carried out.

The precipitated product may be recovered by conventional means. Preferably, the recovery step includes filtering the cooled solution, and drying it under reduced pressure, preferably in vacuum (pressure of less than 100 mmHg).

The method of the present invention preferably yields about 50% to about 80% of Paricalcitol. Preferably, the Paricalcitol prepared according to the method of the present invention has a purity of at least about 98%, preferably a purity of at least about 98.5% and more preferably a purity of at least about 99%.

The present invention further provides a method for preparing a pharmaceutical composition comprising mixing Paricalcitol prepared according to method of the present invention, and a pharmaceutically acceptable carrier. As used herein, the term “pharmaceutical composition” includes tablets, pills, powders, liquids, suspensions, solutions, emulsions, granules, capsules, suppositories, or injection preparations.

The pharmaceutical composition may be prepared in any dosage form such as a compressed granulate in the form of a tablet for example. Also, uncompressed granulates and powder mixes that are obtained by the method of the present invention in the pre-compression steps can be simply provided in a dosage form of a capsule or sachet. Therefore, dosage forms of pharmaceutical formulations prepared by the method of the present invention include solid dosage forms like tablets, powders, capsules, sachets, troches and losenges.

Preferably, the pharmaceutical composition is formulated into pharmaceutical formulations such as conventional dosage forms, including tablets and capsules. Tablets are preferred dosage forms. In addition, the tablets may be coated with an optional cosmetic tablet coating. The dosage form of the present invention may also be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

Preferably, the method of the present invention produces compressed solid dosage forms. There are three well known processes for manufacturing such dosage forms; (i) direct compression, (ii) dry granulation and (iii) wet granulation. There are two well known processes for wet granulation. A wet granulate can be prepared using a mixer and subsequently the wet granulate is dried in order to obtain a dry homogenous granulate. In another method a wet granulate is prepared by spray granulation. In a fluid-bed, spray granulation process, particles and granulate are built up in a fluid bed by spraying a liquid onto fluidized particles. Thus in such process materials are fluidized in the fluid bed dryer and subsequently a solution is sprayed through a nozzle. The choice of processing approach depends upon the properties of the drug and chosen excipients, for example particle size, blending compatibility, density and flowability.

Having described the invention with reference to certain preferred embodiments, other embodiments will become apparent to one skilled in the art from consideration of the specification. The invention is further defined by reference to the following examples describing in detail the preparation of the compound of the present invention. It will be apparent to those skilled in the art that many modifications, both to materials and methods, may be practiced without departing from the scope of the invention.

### EXAMPLES

HPLC method:

| Column: | Hypersyl Gold (250 × 4.6 5 μm) |
|---|---|
| Mobile phase: | (A) water (95%) |
| | (B) acetonitrile (5%) |
| Gradient: | From 0 to 10 min (A) isocraticaly |
| | From 10 to 30 min (B) increases from 0 to 55% |
| | From 30 to 40 min (A) isocraticaly |
| | From 30 to 40 min (B) increases from 55 to 100% |
| Detection: | 252 nm |
| Flow: | 2 mL/min |
| Detection limit: | 0.02% |

### Example 1

### Crystallization of Paricalcitol from Acetone

500 mg of Paricalcitol were dissolved in 75 ml of acetone in a sonicator at 28° C. over a period of 15 minutes. The clear solution was filtered through glass wool into another flask, and the solution was then concentrated by evaporation, until the volume was 57.5 ml acetone (control by weight). The solution was cooled to −18° C., and the temperature was maintained at −18° C. for 20 hours. The crystals were filtered and washed with 20 ml of cold (−18° C.) acetone, then dried at high vacuum in an oven at 28° C. for 22 hours to obtain a yield of 390 mg (purity of 98.54%).

### Example 2

### Crystallization of Paricalcitol from Acetone+Water

540 mg of Paricalcitol were dissolved in 81 ml of acetone in a sonicator at 28° C. over a period of 15 minutes. The clear solution was filtered through glass wool into another flask, and 8 ml water was added. The solution was then concentrated by evaporation to a volume of 54 ml of acetone (control by weight). The solution was cooled to −18° C., and that temperature was maintained for 16 hours The crystals were filtered and washed with 20 ml of cold (−18° C.) acetone, and then dried at high vacuum in an oven at 28° C. for 6 hours to obtain a yield of 300 mg (purity of 99.79%).

### Example 3

### Crystallization of Paricalcitol from Ethyl Acetate

520 mg of Paricalcitol were dissolved in 100 ml of Ethyl acetate in a sonicator at 28° C. over a period of 15 minutes. The clear solution was filtered through glass wool into another flask, and the solution was then concentrated by evaporation to a volume of 86 ml of Ethyl acetate (control by weight). The solution was cooled to −18° C., and that temperature was maintained for 20 hours. The crystals were filtered and washed with 20 ml of cold (−18° C.) Ethyl acetate, then dried at high vacuum in an oven at 28° C. for 20 hours to obtain a yield of 360 mg (purity of 98.46%).

### Example 4

### Crystallization of Paricalcitol from Ether-Acetone

1.25 g of Paricalcitol were dissolved in 290 ml of diethyl ether-acetone solution (1:2) with stirring at 34° C. over a period of 30 minutes. The solution was then concentrated by evaporation to a total weight of about 150 g. The solution was cooled to −18° C., and that temperature was maintained for 4 hours. The crystals were filtered and washed with 20 ml of cold acetone (−18° C.), then dried at high vacuum in an oven at 30° C. for 1 hour to obtain a yield of 920 mg.

### Example 5

### Crystallization of Paricalcitol from Ether-Methyl Formate-CH₃CN-EtOH

1.07 g of Paricalcitol were dissolved in a mixture of 150 ml Ether, 150 ml Methyl formate, 100 ml CH₃CN, and 20 ml EtOH. The solution was cooled to 0° C., and seeded with crystals of Paricalcitol, cooled to −45° C., and stirred at −45° C. for 1 hour. The crystals were filtered, and then dried at high vacuum in an oven at 28° C. for 2 hours to obtain a yield of 630 mg (purity of 99.38%).

### Example 6

### Crystallization of Paricalcitol from Tert-Butanol

100 mg of Paricalcitol were dissolved in 17 ml of tert-Butanol with stirring at 30° C. over a period of 30 minutes. The solution was then concentrated by evaporation at 30° C. to a volume of about 11 ml tert-Butanol (control by weight). The solution was cooled to 25° C., and stirred at that temperature for 1 hour. The crystals were filtered and then dried at high vacuum in an oven at 28° C. for 20 hours to obtain a yield of 60 mg (purity of 99.63%).

### Example 7

### Crystallization of Paricalcitol from Acetone

1.35 g Paricalcitol were dissolved in 270 ml Acetone, at 32° C., with stirring, during 15 min. Then, the solution was filtered through glass wool to another flask and the solvent was carefully evaporated, under reduced pressure at 32° C., until a volume of 218 ml acetone.

Then, the solution was cooled to 10° C. and the solution was seeded with 18 mg Paricalcitol then cooled to −18° C. and stirred at −18° C., at 200 rpm for 16 hours. The obtained crystalline material was filtered, washed with 20 ml cold (−18° C.) acetone, and dried at 28° C. under vacuum (P˜2 mmHg) for 6 hours, to give 900 mg cryst. Paricalcitol.

### Example 8

### Crystallization of Paricalcitol from Acetone

2.35 g Paricalcitol were dissolved in 353 ml Acetone, at 28° C., in the sonicator, during 15 min. Then, the solution was filtered through glass wool to another flask which was put, in the Lauda at 22° C.

Then, stirring was started and the flask was cooled to −18° C. during 12 hours and continue stirring at −18° C., for another 6 hours.

The obtained crystalline material was filtered, washed with 20 ml cold (−18° C.) acetone, and dried at 28° C. under vacuum (P˜2 mmHg) for 6 hours, to give 1.81 g cryst. Paricalcitol.

### Example 9

### Crystallization of Paricalcitol from Ethyl Acetate+Water

0.40 g Paricalcitol was dissolved in 80 ml ethyl acetate, in the sonicator, at 28° C., during 10 min. Then, the solution was filtered through glass wool to another flask, and 6.5 ml water was added. The solvent was carefully evaporated, under reduced pressure at 32° C., until a volume of 66 ml ethyl acetate (=165 volumes, control by weight). Then, the flask was put at −18° C. for 16 hours.

The obtained crystalline material was filtered, washed with 30 ml cold (−18° C.) ethyl acetate, and dried at 28° C. under vacuum (P˜2 mmHg) for 22 hours, to give 0.23 g cryst. Paricalcitol. (purity of 98.88%)

### Example 10

### Crystallization of Paricalcitol from CH₃CN (Acetonitrile)

1.01 g Paricalcitol were dissolved in 200 ml CH₃CN, at 30° C., in the sonicator, during 30 min. Then, the solution was filtered through glass wool to another flask which was put, in the Lauda at 22° C.

Then, stirring was started and the flask was cooled to −18° C. and continue stirring at −18° C., for 18 hours.

The obtained crystalline material was filtered, washed with 20 ml cold (−18° C.) CH₃CN, and dried under vacuum (P˜2 mmHg) at 28° C. for 20 hours, to give 0.6 g cryst. Paricalcitol.

### Example 11

### Crystallization of Paricalcitol from CH₃CN+Water

0.4 g Paricalcitol were dissolved in 160 ml solution of 5% water in CH₃CN, at 30° C., in the sonicator, during 15 min. Then, the solution was filtered through glass wool to another flask which was put, in the Lauda at 22° C.

Then, stirring was started and the flask was cooled to −18° C. and continue stirring at −18° C., for 18 hours.

The obtained crystalline material was filtered, washed with 20 ml cold (−18° C.) CH₃CN, and dried under vacuum (P˜2 mmHg) at 28° C. for 20 hours, to give 0.28 g cryst. Paricalcitol.

1. A method for purifying Paricalcitol comprising the steps of
a) dissolving Paricalcitol in a solvent;
b) cooling the solution to form a precipitate; and
c) recovering precipitate.

2. The method according to claim 1, wherein the solvent is selected from the group consisting of a C₂-C₆ ether, a C₂-C₄ ester, a mixture of C₂-C₄ ester/H₂O, a C₃-C₅ ketone, a mixture of C₃-C₅ ketone/H₂O, a C₁-C₄ alcohol, a mixture of C₂-C₆ ether/C₃-C₅ ketone, a mixture of C₂-C₆ ether/C₂-C₄ ester, a mixture of C₂-C₆ ether/C₁-C₄ alcohol, acetonitrile, a mixture of acetonitrile/H₂O, and mixtures thereof.

3. The method according to claim 2, wherein the solvent is selected from the group consisting of tert-butanol, acetone, acetone/H₂O, diethyl ether, ethyl acetate, ethyl acetate/H₂O, diethyl ether/acetone, acetonitrile, acetonitrile/H₂O, and mixtures thereof.

4. The method according to claim 1, wherein Paricalcitol and the solvent in step a) are in a ratio of about 1:150 to about 1:450 g Paricalcitol/ ml solvent.

5. The method according to claim 4, wherein the ratio is about 1:150 to about 1:200.

6. The method according to claim 1, wherein dissolving Paricalcitol in a solvent is carried out at a temperature of about 25° C. to about 40° C.

7. The method according to claim 6, wherein the temperature is about 28° C. to about 34° C.

8. The method according to claim 1, further comprising the step of filtering the solution obtained in step a) after dissolving Paricalcitol in a solvent.

9. The method according to claim 1, wherein the solution is cooled to a temperature of about −45° C. to about −10° C.

10. The method according to claim 9, wherein the solution is cooled to a temperature of about −20° C. to about −15° C.

11. The method according to claim 10, wherein the solution is cooled to a temperature of about −18° C.

12. The method according to claim 3, wherein when the solvent is tert-butanol the solution is cooled to a temperature of about 25° C. to about 27° C.

13. The method according to claim 1, wherein the solution is cooled at a rate of not more than about 8° C. per hour.

14. The method according to claim 1, wherein the solution is cooled for a period of about 1 to about 24 hours.

15. The method according to claim 14, wherein the period is about 15 to about 24 hours.

16. The method according to claim 15, wherein the period is about 16 to about 20 hours.

17. The method according to claim 1, wherein dissolving Paricalcitol in a solvent is carried out in a sonicator.

18. The method according to claim 1, the method further comprises concentrating the solution from step a) before cooling the solution.

19. The method according to claim 18, wherein the solution is concentrated to about 0.5 to about 0.9 times its original volume.

20. The method according to claim 19, wherein the solution is concentrated to about 0.6 to about 0.8 times its original volume.

21. The method according to claim 19, wherein Paricalcitol and the solvent are in a ratio of about 1:100 to about 1:120 g Paricalcitol: ml solvent.

22. The method according to claim 1, wherein when the method further comprises seeding the solution with crystals of Paricalcitol either before or during the step of cooling the solution.

23. Paricalcitol prepared according to the method of claim 1, wherein the Paricalcitol has a purity of at least about 98%.

24. The Paricalcitol according to claim 23, wherein the Paricalcitol has a purity of at least about 99%.

25. A method of preparing a pharmaceutical composition of Paricalcitol comprising mixing Paricalcitol prepared according to claim 1 with a pharmaceutically acceptable carrier.
